(19) United States
(12) Patent Application Publication
(10) Pub. No.: US 2010/0074886 A1
(43) Pub. Date: Mar. 25, 2010

(54) CCR2 ANTAGONISTS FOR TREATMENT OF FIBROSIS

(76) Inventors: Anuk Das, Berwyn, PA (US); Lynne Murray, King of Prussia, PA (US); Fahrat Syed, Audubon, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/442,404

(22) PCT Filed: Oct. 5, 2007
(86) PCT No.: PCT/US2007/080542

§ 371 Date: Nov. 18, 2009

Related U.S. Application Data
(60) Provisional application No. 60/828,253, filed on Oct. 5, 2006.

(51) Int. Cl. A61K 39/395; A61K 38/16; A61K 31/404; A61P 7/00

(52) U.S. Cl. 424/130.1; 514/12; 514/415

(57) ABSTRACT

Anti-MCP-1/CCR2 antagonist therapy is provided for the control or reversal of fibrosis related diseases, including, e.g., but not limited to MCP-1/CCR2 antagonist therapy for the modulation of profibrotic markers associated with fibrotic processes including collagen matrix deposition and alveolar collapse.

### BACKGROUND OF THE INVENTION

1. Field of the Invention

The invention relates to methods of the using and antagonist of CCL2 binding to CCR2, such as an anti-CCL2 antibody, for the prevention and control of pulmonary fibrosis, particularly usual interstitial pneumonia.

2. Description of the Related Art

Usual interstitial pneumonia (UIP) is a chronic debilitating interstitial lung disease that is characterized and routinely diagnosed by the presence of honeycombing within the lung. The honeycombing arises because of an increase in collagen deposition, therefore reducing the elasticity within the lung and causing retraction and ultimate collapse of normal alveolar structure. The extent of honeycombing and fibrosis is very heterogeneous within the lung, where dense areas of excess collagen often adjoin normal lung parenchyma or interstitial tissue rich in monocytic infiltrates. Most patients have moderate to advanced clinical disease at the time of diagnosis and deteriorate despite treatment.

The CXC chemokine family is a pleiotropic family of cytokines that are involved in promoting the trafficking of various leukocytes, in regulating angiogenesis and vascular remodeling, and in promoting the mobilization and trafficking of mesenchymal progenitor cells such as fibrocytes which are circulating mesenchymal progenitor cells (also known as fibrocytes) in pulmonary fibrosis.

Within the normal lung, a large pool of resident fibroblasts continuously generate and breakdown collagen, thereby allowing the lung to remodel following infection, inflammation or other pathophysiology, and after injury. Fibroblasts generate collagen in response to various growth factors such as TGFb1. Furthermore, TGFb1 induces fibroblast differentiation into myofibroblasts, which are commonly found in UIP lung tissue. Myofibroblasts are differentiated cells capable of generating collagen as well as having alpha-smooth muscle actin and therefore contractile properties. Myofibroblasts may therefore potentially contribute to alveolar collapse. In normal course of wound healing, fibroblasts generate collagen and growth factors to direct wound closing. Once tissue architecture is restored, fibroblast collagen generation decreases and the cells go through apoptosis, thus preventing excess scar formation. There is pronounced fibroblast proliferation found in the lungs of UIP patients. Therefore UIP fibroblasts persist at sites of fibrosis, continuously adding to the aberrant excessive collagen deposition. UIP patients are among common recipients of lung transplants and, in these patients, the transplant may eventually become fibrotic.

Previous work has shown phenotypic differences in fibroblasts isolated from sites of fibrosis compared to fibroblasts isolated from non-fibrotic tissue. For example, fibroblasts isolated from the lungs of UIP patients have increased expression of IL-13 receptor subunits. CCR2 is expressed on lung fibrocytes and CCR2 regulates both recruitment and activation of these cells after respiratory injury. Inhibition of CCR2 signaling in vivo through either receptor knockout mice or ligand neutralization results in less collagen deposition in multiple animal models of fibrosis. CCL2 (CC-chemokine ligand 2, Monocyte Chemoattractant Protein 1, MCP1) binds to CCR2. CCR2 is predominantly expressed on monocytes, epithelial cells and endothelial cells. Increased levels of MCP1 have been described in patients with UIP. Ligands for the CCR2 receptor in the mouse include CCL2 (also known as JE or monocyte chemoattractant protein [MCP]-1), CCL7 (MCP-3) and CCL12 (MCP-5), thus, assumptions based on murine model data may not accurately reflect the human pulmonary environment with respect to chemokine and chemokine receptor distributions.

Monocyte chemoattractant protein 1 (MCP-1, CCL2, ligand for CCR2, GenBank NP_{—}002973), an 8.6 kDa protein containing 76 amino acid residues, is a member of the chemokine-beta (or C—C) family of cytokines. MCP-1 is expressed by a variety of cell types including monocytes, vascular endothelial cells, smooth muscle cells, glomerular mesangial cell, osteoblastic cells, and human pulmonary type-2-like epithelial cells. It is believed that MCP-1 plays an active role in the initiation and progression of inflammatory diseases, by promoting monocyte influx and subsequent activation in tissues. MCP-1 is chemotactic for monocytes but not neutrophils. It can induce the proliferation and activation of killer cells known as CHAK (CC-chemokine activated killer), which are similar to cells activated by IL-2. It regulates the expression of cell surface antigens (CD11c, CD11b) and the expression of cytokines IL1 and IL6. MCP-1 is a potent activator of human basophils, inducing the degranulation and the release of histamines.

Thus, there is a need in the medical art for methods to monitor for and manage patients displaying the hallmarks of the pathophysiology leading to pulmonary insufficiency known as UIP and to be able avoid the need for lung transplantation and, at a minimum, enhance the safety and survival in allograft utilization, and to understand and remedy the pathological actions of MCP-1 therein.

### SUMMARY OF THE INVENTION

The present invention provides a method of preventing, slowing, or reversing fibrosis in subject, comprising contacting or administering a composition comprising an effective amount of at least one isolated CCR2 antagonists which prevents the biological functions or bioactivity associated with CCR2, its isoforms or variants including CCR2A or CCR2B, in cells that display the receptor as defined herein or antagonists which bind MCP-1/CCL2 or CCR2 or which prevent the binding of CCR2 with its cognate ligand(s) and thereby inhibit CCR2 biological functions in the cell, tissue, organ of the mammalian subject. In one aspect of the method of the invention the subject is a patient having an interstitial pathology and alveolar fibrosis related to interstitial idiopathic pneumonia, more specifically, the patient is diagnosed with usual interstitial pneumonia.

The method of the invention may be practiced with a CCR2 antagonists which prevents the biological functions or bioactivity associated with CCR2, its isoforms or variants including CCR2A or CCR2B, in cells that display the receptor as defined herein. In one aspect of the invention, CCR2 antagonists include antibodies, synthetic or native sequence peptides and small molecule antagonists, which bind MCP-1/CCL2 or CCR2 or which prevent the binding of CCR2 with its cognate ligand(s) and thereby inhibit CCR2 biological functions.

Also provided is a method for diagnosing an MCP-1 related interstitial pathology and alveolar fibrosis related to interstitial idiopathic pneumonia in a cell, tissue, organ or animal, comprising

In an embodiment, the ligand binding portions of the antibody comprise SEQ ID NO: 27 and 28. In one aspect, the present invention provides at least one isolated mammalian anti-MCP-1 antibody, comprising at least one variable region comprising SEQ ID NO: 27 or 28.

In another aspect, the present invention provides at least one isolated mammalian anti-MCP-1 antibody, comprising either (i) all of the heavy chain complementarity determining regions (CDR) amino acid sequences of ID NOS: 6, 7 and 9; or (ii) all of the light chain CDR amino acids sequences of SEQ ID NOS: 13, 14, and 16.

The present invention further provides at least one anti-MCP-1 antibody method or composition, for administering a therapeutically effective amount to modulate or treat at least one MCP-1 related condition in a cell, tissue, organ, animal or patient and/or, prior to, subsequent to, or during a related condition, as known in the art and/or as described herein. In another aspect, the present invention provides at least one isolated mammalian anti-MCP-1 antibody, comprising either (i) all of the heavy chain complementarity determining regions (CDR) amino acid sequences of SEQ ID NOS: 6, 7 and 8 or 9; or (ii) all of the light chain CDR amino acids sequences of SEQ ID NOS: 13, 14 and 15 or 16.

The present invention also provides at least one composition, device and/or method of delivery of a therapeutically or prophylactically effective amount of at least one anti-MCP-1 antibody, according to the present invention.

The present invention further provides any invention described herein.

### BRIEF DESCRIPTION OF THE FIGURES

FIG. 1 shows a column graph where each column represents the fold increase in the expression of a genes associated with fibrosis in UIP fibroblasts in comparison to fibroblasts isolated from non-fibrotic lung tissue (normalized to a value of 1): aSMA: PCOL1; PCOL3; CTGF; TGFβ-1; TGFβR1; TGFβR2; IL13Ra1; IL13Ra2.

FIG. 2 is a column graph showing the effect of TGFβ1, PDGF and CCL2 on aSMA expression by fibroblasts derived from non-fibrotic and fibrotic lung tissue.

FIG. 3 is two column graphs showing the effect of TGFβ1, PDGF and CCL2 on procollagen I (A) and procollagen III (B) gene expression by fibroblasts derived from non-fibrotic and fibrotic lung tissue.

FIG. 4 is a column graph showing the effect of TGFβ1, PDGF and CCL2 on TGFβ1 gene expression by fibroblasts derived from non-fibrotic and fibrotic lung tissue.

FIG. 5 is a column graph showing the effect of TGFβ1, PDGF and CCL2 on CTGF gene expression by fibroblasts derived from non-fibrotic and fibrotic lung tissue.

FIG. 6 is two column graphs showing the effect of TGFβ1, PDGF and CCL2 on TGFβRI (A) and TGFβRII (B) gene expression by fibroblasts derived from non-fibrotic and fibrotic lung tissue.

FIG. 7 is two column graphs showing the effect of TGFb1, PDGF and CCL2 on IL13Ra1 (A) and IL13Ra2 (B) gene expression by fibroblasts derived from non-fibrotic and fibrotic lung tissue.

### BRIEF DESCRIPTION OF THE SEQUENCE LISTING

| SEQ ID | |
|---|---|
| NO: | Description |
| 1 | Human MCP-1 (CCL2) and variants used to select |
| | anti-MCP-1 binders |
| 2 | VH1A heavy chain variable sequence: FR1, CDR1, FR2, CDR2 |
| | variants, FR3, CDR3, FR4 |
| 3 | VH3 Heavy chain variable sequence: FR1, CDR1, FR2, CDR2 |
| | variants, FR3, CDR3, FR4 |
| 4 | Kappa3 light chain variable sequence: FR1, CDR1, FR2, CDR2, |
| | FR3, CDR3 variants, FR4 |
| 5 | Lambda3 light chain variable sequence: FR1, CDR1, FR2, |
| | CDR2, FR3, CDR3 variants, FR4 |
| 6 | VH1A CDR1 All MOR03471 |
| 7 | VH1A CDR2 3781, 3790, CNTO 888 |
| 8 | VH1A CDR2 3899 |
| 9 | VH1A CDR3 All MOR03471 |
| 10 | VH3 CDR1 All MOR03548 |
| 11 | VH3 CDR2 3744, 3747 |
| 12 | VH3 CDR3 All MOR03548 |
| 13 | Kappa3 CDR1 All MOR03471 |
| 14 | Kappa3 CDR2 All MOR03471 |
| 15 | Kappa3 CDR3 3781 |
| 16 | Kappa3 CDR3 3790, CNTO888 |
| 17 | Kappa3 CDR3 3899 |
| 18 | Lamda3 CDR1 All MOR03548 |
| 19 | Lamda3 CDR2 All MOR03548 |
| 20 | Lamda3 CDR3 3744 |
| 21 | Lamda3 CDR3 3747 |
| 22 | VH1A CDR2 Variants |
| 23 | VH3 CDR2 Variants |
| 24 | Lk CDR3 Variants |
| 25 | Lλ CDR3 Variants |
| 26 | HC CDR1 Variants |
| 27 | CNTO888 Heavy Chain Variable Region |
| 28 | CNTO888 Light Chain Variable Region |

### DETAILED DESCRIPTION OF THE INVENTION

### Abbreviations

Abs antibodies, polyclonal or monoclonal; Ig immunoglobulin; Mab monoclonal antibody; V variable domain of an antibody; C constant domain of an antibody; H heavy chain of an antibody; L light chain of an antibody; HRCT high-resolution computed tomography; PDGF-AB platelet-derived growth factor alpha/beta; CTGF: connective tissue growth factor; CXC chemokine of the CXC subclass; aSMA alpha-smooth muscle actin; PCOL1 procollagen I; PCOL3: procollagen III; TGFβ1 transforming growth factor beta-1; TGFβR1 TGFβ receptor type I; TGFβR2: TGFβ receptor type II; IL13Ra1: Interleukin-13 receptor alpha 1 subunit; IL13Ra2: Interleukin-13 receptor alpha 2 subunit.

### DEFINITIONS

The term “antibody” herein is used in the broadest sense. As used herein, an “antibody” includes whole antibodies and any antigen binding fragment or a single chain thereof. Thus, the antibody includes any protein or peptide containing molecule that comprises at least a portion of an immunoglobulin molecule, such as but not limited to at least one complementarity-determining region (CDR) of a heavy or light chain or a ligand binding portion thereof, a heavy chain or light chain variable region, a heavy chain or light chain constant region, a framework (FR) region, or any portion thereof, or at least one portion of a binding protein, which can be incorporated into an antibody of the present invention. The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments to a preselected target. Examples of binding fragments encompassed within the term “antigen binding portion” of an antibody include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. 1988 Science 242:423-426, and Huston et al. 1988 Proc. Natl. Acad. Sci. USA 85:5879-5883. Such single chain antibodies are also intended to be encompassed within the term “antigen-binding portion” of an antibody. Antibody fragments are obtained using conventional techniques known to those with skill in the art, and the fragments are screened for utility in the same manner as are intact antibodies.

By “CCR2” is meant human CCR2A (MCP-1RA, NP_{—}000638) and/or human CCR2B (MCP-1RB, NP_{—}000639) and to proteins having an amino acid sequence which is the same as that of a naturally occurring or endogenous corresponding mammalian CCR2 protein (e.g., recombinant proteins). CCR2A, isoform A, has distinct C-terminus and is 14 amino acids longer than CCR2B, isoform B, due to alternative splicing in the coding region that results in a frameshift and use of a downstream stop codon (Charo, et al. 1994. Proc. Natl. Acad. Sci. U.S.A. 91 (7): 2752-2756). CCR2, as defined herein, includes mature receptor protein, polymorphic or allelic variants, and isoforms of a mammalian CCR2 (e.g., produced by alternative splicing or other cellular processes), and modified or unmodified forms of the foregoing (e.g., glycosylated, unglycosylated). Such proteins can be recovered or isolated from a source which naturally produces mammalian CCR2, for example.

A “CCR2 antagonist” prevents the biological functions or bioactivity associated with CCR2A or CCR2B in cells that display CCR2A or CCR2B or other isoforms or variants as defined herein. Antagonists included within the scope of the present invention include antibodies, synthetic or native sequence peptides and small molecule antagonists, which bind MCP-1/CCL2 or CCR2 or which prevent the binding of CCR2 with its cognate ligand(s) and thereby inhibit CCR2 biological functions. Thus, an inhibitor refers to substances including antagonists which bind receptor (e.g., an antibody, a mutant of a natural ligand, small molecular weight organic molecules, other competitive inhibitors of ligand binding), and substances which inhibit receptor function without binding thereto (e.g., an anti-idiotypic antibody).

By “usual interstitial pneumonia” or “UIP” is also known clinically and histologically as “idiopathic pulmonary fibrosis” or “IPF” and “cryptogenic fibrosing alveolitis”. It is the most common of the six histologic subtypes of idiopathic interstitial pneumonia (IIP). Other IIPs are: nonspecific interstitial pneumonia (NSIP), bronchiolitis obliterans organizing pneumonia (BOOP); respiratory bronchiolitis-associated interstitial lung disease (ILD); desquamative interstitial pneumonia; and acute interstitial pneumonia (AIP).

By “MCP-1” is meant the 76 amino acid sequence referenced in NCBI record accession No. NP_{—}002973 and variously known as MCP (monocyte chemotactic protein), SMC-CF (smooth muscle cell chemotactic factor), LDCF (lymphocyte-derived chemotactic factor), GDCF (glioma-derived monocyte chemotactic factor), TDCF (tumor-derived chemotactic factors), HC11 (human cytokine 11), MCAF (monocyte chemotactic and activating factor). The gene symbol is SCYA2, the JE gene on human chromosome 17, and the new designation is CCL2 (Zlotnik, Yoshie 2000. Immunity 12:121-127). JE is the mouse homolog of human MCP-1/CCL2.

An MCP-1 antagonist small molecule refers to any suitable chemical compound that inhibits MCP-1 activity and can be used a potential therapeutic. Such compounds are known in the art, such as indole derivatives, cyclic amine derivatives, ureido derivatives, heterocyclics, anilides, and functional pyrroles with the ability to block CCL2 binding to CCR2B, and/or inhibition of CCR1 or CCL2 itself, as disclosed in PCT publications WO 9905279 (1999), WO 9916876 (1999), WO 9912968, WO 9934818, WO 9909178, WO 9907351, WO 9907678, WO 9940913, WO 9940914, WO 0046195, WO 0046196, WO 0046197, WO 0046198, WO 0046199, WO 9925686, WO 0069815, WO 0069432, WO 9932468, WO 9806703, WO 9904770, WO 99045791, each of which is entirely incorporated herein by reference.

As used herein “treating” used in this invention means both treatments that comprise “controlling” and “reversing” the functional or histological signs of chronic rejection.

Mammals which maybe treated in the present invention include livestock mammals such as cows, houses, etc., domestic animals such as dogs, cats, rats, etc. and humans, preferably humans.

### Citations

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2006; Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2006; Colligan et al., Current Protocols in Protein Science, John Wiley & Sons, NY, N.Y., (1997-2006.

### Usual Interstitial Pneumonia (UIP) and Idiopathic Pulmonary Fibrosis Compounds of the Invention

MCP-1 is known to bind and signal through the chemokine receptor CCR2. CCR2 is a seven trans-membrane-spanning G-protein-coupled receptor expressed on many cells including monocytes, T-cells, B-cells, and basophils. Two MCP-1 specific receptors, CCR2A and CCR2B, have been cloned which signal in response to nanomolar (nM) concentrations of MCP-1. CCR2A (CC—CKR2A) and CCR2B (CC—CKR2A) represent two cDNAs that encode two MCP-1-specific receptors with alternatively spliced carboxyl tails. MCP-1 binds to both isoforms with high affinity MCP-1 induces calcium flux in cells expressing CCR2B but not in cells expressing CCR2A. 5-fold less MCP-1 induces chemotaxis in cells expressing CCR2B compared to cells expressing CCR2A.

Other proteins with certain functional and sequence homology to human MCP-1 are known. Especially similar to MCP-1 (GenBank NP_{—}002973) are MCP-2 (GenBank NP_{—}005614) and eotaxin (GenBank P_{—}51671); MCP-2 having 61.8 percent and eotaxin-1 having 63.2 percent sequence identity to MCP-1. The range of activities and spectrum of involvement of these proteins in human homeostatic mechanisms and pathology is not as well understood for the homologs of MCP-1. For example, MCP-2 (renamed CCL8) is related closely to MCP-1 and MCP-3 (renamed CCL7, Genbank NP_{—}006264) and uses both CCR1 as well as CCR2B as its functional receptors. MCP-3 binds to a receptor designated D6. MCP-3 also binds to CCR10 and CCR1. The MCP-3 protein (97 amino acids) sequence shows 74 percent identity with MCP-1 and 58 percent homology with MCP-2. Secreted MCP-3 differs from MCP-1 in being N-glycosylated. MCP-4 (renamed CCL13, Genbank NP_{—}005399) shares 56-61 percent sequence identity with the three known monocyte chemotactic proteins and is 60 percent identical with Eotaxin-1. The functions of MCP-4 appear to be highly similar to those of MCP-3 and Eotaxin. Like MCP-3, MCP-4 is a potent chemoattractant for monocytes and T-lymphocytes. It is inactive on neutrophils. On monocytes, MCP-4 binds to receptors that recognize MCP-1, MCP-3, RANTES (CCL5), and eotaxin, the CCR1 and CCR3 receptors, and shows full cross-desensitization with eotaxin-1. MCP-5 is murine CC-chemokine and related most closely to human MCP-1 (66% amino acid identity). The gene symbol for MCP-5 is SCYA12 (renamed CCL12). Cells transfected with the chemokine receptor CCR2 have been shown to respond to MCP-5. General information on cytokines and chemokines is available on the world-wide internet and for the current classification system, Zlotnik A., Yoshie O. 2000. Chemokines: a new classification system and their role in immunity. Immunity 12:121-127.

The forgoing discussion serves to emphasize that an antagonist may prevent the biological function of CCR2 binding by either direct action on CCR2 or one of its ligands, CCL2, CCL7, CCL8. In one embodiment of the invention, the antagonist binds to MCP-1/CCL2 and neutralizes its ability to bind to CCR2.

Anti-CCR2 antibodies are disclosed in U.S. Pat. No. 6,084,075, U.S. Pat. No. 6,458,353 and U.S. Pat. No. 6,696,550. In one embodiment of the method of the invention, a method of inhibiting the biological interaction of a cell bearing mammalian CCR2 with a chemokine, comprises contacting said cell with an effective amount of an antibody or functional fragment thereof which binds to CCR2 or a portion of said receptor. In one embodiment, the antibody is monoclonal antibody (mAb) LS132.1D9 (1D9) or an antibody, which can compete with 1D9 for binding to human CCR2 or a portion of human CCR2. Functional fragments of the foregoing antibodies are also envisioned.

Antibodies capable of binding MCP-1 have been reported: JP9067399 discloses an antibody obtained from isolated blood cells and JP05276986 discloses a hybridoma secreting an IgM anti-human MCP-1. More recently, antibodies capable of binding a plurality of beta-chemokines including MCP-1 were disclosed (WO03048083) and an MCP-1 binding antibody which also binds eotaxin (US2004/0047860). Antibodies which selectively bind and neutralize mouse homologs of human MCP-1/CCL2 or human MCP-1/CCL2 are disclosed in applications co-pending patent applications U.S. Ser. No. 11/170,453 and 60/682,654 the contents and teachings of which are incorporated herein by reference.

In one embodiment of the invention, the CCR2 antagonist is the anti-human MCP-1/CCL2 antibody designated C775 which can be produced by a cell line designated C1142 as disclosed in applications co-pending patent applications U.S. Ser. No. 11/170,453, variants such as humanized or reshaped forms, truncated forms, or binding fragments thereof as defined herein. In another embodiment, the CCR2 antagonist is the anti-human MCP-1/CCL2 antibody designated CNT0888 variants, truncated forms, or binding fragments thereof as defined herein and as disclosed in applications co-pending patent applications WO2006125202, the contents and teachings of which are incorporated herein by reference.

In one embodiment, the MCP-1 antibody comprising both heavy chain and light chain variable regions comprising SEQ ID NOS: 27 and 28. The antibody can comprise at least one heavy chain variable region and at least one light chain variable region, said antibody comprising all of the heavy chain and light chain complementarity determining region (CDR) amino acid sequences of SEQ ID NOS: 6, 7, 9, 13, 14, and 16. The antibody comprises at least one variable region comprising at least one heavy chain and at least one light chain, said MCP-1 antibody comprising both heavy chain and light chain variable regions comprising SEQ ID NOS: 27 and 28. The antibody can comprise at least one heavy chain variable region and at least one light chain variable region, said antibody comprising all of the heavy chain and light chain complementarity determining region (CDR) amino acid sequences of SEQ ID NOS: 6, 7, 9, 13, 14, and 16. The antibody can comprise at least one heavy chain or light chain CDR having the amino acid sequence of at least one of SEQ ID NOS: 6, 7, 9, 13, 14, and 16. The antibody can alternatively comprise a heavy chain or light chain variable region of at least one of SEQ ID NO: 2-5 further comprising a complementarity determining region (CDR) of a heavy or light chain or a ligand binding portion thereof selected from the group consisting of SEQ ID NO: 6-26; and, optionally functionally associated with a framework region, further optionally comprising at least CH1, hinge, CH2, or CH3 of an human immunoglobulin.

MCP-1/CCL2 truncations, variants, mutant proteins or “muteins” having the ability to bind CCR2 and have antagonistic activity may also be used to practice the method of the invention. Variants of homodimer-forming chemokines, such as CCL2, having a single amino acid substitution in the dimerization interface that alters the pattern of hydrogen bonds, so as to result in an obligate monomer that binds to the receptor and has agonistic properties in vitro but which can antagonize natural chemokines and have anti-inflammatory activity in vivo as taught in WO05037305A1 are among the variants useful in practicing the present invention. A peptide antagonist of MCP 1, is the truncated MCP-1 (9-76), which was shown both to prevent disease onset and to reduce disease symptoms in a mouse model of arthritis (Jiang-Hong Gong, et al., J. Exp. Med. 1997, 186:131).

### Modulation of CCR2/CCL2 Expression

An alternate method of antagonizing the interaction of CCR2 with its ligands, is by knocking down the expression of the CCR2 or its ligands, especially MCP-1/CCL2, using e.g. methods of RNA silencing. Thus, in another embodiment, compounds useful in practicing the method of the invention are nucleic acids, including oligonucleotides and polynucleotides in sense or antisense orientation, and single or double stranded nucleic acid molecules (e.g., siRNA) that target MCP-1 sequences and interfere with MCP-1 gene expression or that target CCR2 and interfere with CCR2 gene expression.

Gene expression can be modulated in several different ways, including by the use of siRNAs, shRNAs, antisense molecules and DNAzymes. SiRNAs and shRNAs both work via the RNAi pathway and have been successfully used to suppress the expression of genes. RNAi was first discovered in worms and the phenomenon of gene silencing related to dsRNA was first reported in plants by Fire and Mello (Fire et al., 1998. Nature 391: 806) and is thought to be a way for plant cells to combat infection with RNA viruses. In this pathway, the long dsRNA viral product is processed into smaller fragments of 21-25 bp in length by a DICER-like enzyme and then the double-stranded molecule is unwound and loaded into the RNA induced silencing complex (RISC). A similar pathway has been identified in mammalian cells with the notable difference that the dsRNA molecules must be smaller than 30 bp in length in order to avoid the induction of the so-called interferon response, which is not gene specific and leads to the global shut down of protein synthesis in the cell.

Synthetic siRNAs can be designed to specifically target one gene and they can easily be delivered to cells in vitro or in vivo. ShRNAs are the DNA equivalents of siRNA molecules and have the advantage of being incorporated into the cells' genome and then being replicated during every mitotic cycle.

DNAzymes have also been used to modulate gene expression. DNAzymes are catalytic DNA molecules that cleave single-stranded RNA. They are highly selective for the target RNA sequence and as such can be used to down-regulate specific genes through targeting of the messenger RNA.

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

siRNAs are double stranded RNAs that include the target sequence and its complement. Two uridine residues are added to the 3′ end of the RNAs (Elbashir et al. 2001 Nature 411:494-498).

RNA interference (RNAi) is now being used routinely in mammalian cells to study the functional consequences of reducing the expression of specific genes. RNAi is induced by transfecting small interfering RNAs (siRNAs), comprising double-stranded RNA molecules ˜21 nt in length with 2 nt 3′ overhangs (Elbashir et al. 2001 supra), or hairpin-forming 45-50mer (shRNA) molecules (Paddison, P J, et al., 2002. Genes & Development 16:948-958), that are complementary to the gene of interest. When transfected into mammalian cells, siRNA expression plasmids and have been shown to reduce the levels of both exogenous and endogenous gene products. Although they require more effort to prepare than chemically synthesized or in vitro transcribed siRNAs, the siRNA vectors can provide longer term reduction in target gene expression when coexpressed with a selectable marker (Brummelkamp, T R, et al., 2002. Science 296:550-553).

### Non-Protein, Non-Oligonucleic Acid Antagonists

Small molecule drugs and peptidomimetics can also be antagonists of CCR2. For example, WO04069809, WO04069810, WO05118574, WO06015986 teach mercaptoimidazoles as CCR2 receptor antagonists. Other small molecules exhibiting the desired biological properties can be selected by screening using methods such as those described herein and will have the property of preventing chronic rejection and prolonging graft survival.

### Methods of Making Antibodies

CCR2 antagonist antibodies of the present invention can be optionally produced by a variety of techniques, including the standard somatic cell hybridization technique (hybridoma method) of Kohler and Milstein (1975) Nature 256:495. In the hybridoma method, a mouse or other appropriate host animal, such as a hamster or macaque monkey, is immunized as described herein to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The CCR2 antagonistic antibody can also be optionally generated by immunization of a transgenic animal (e.g., mouse, rat, hamster, non-human primate, and the like) capable of producing a repertoire of human antibodies, as described herein and/or as known in the art. Cells that produce, e.g. a human anti-MCP-1 antibody can be isolated from such animals and immortalized using suitable methods, such as the methods described herein.

The use of transgenic mice carrying human immunoglobulin (Ig) loci in their germline configuration provide for the isolation of high affinity fully human monoclonal antibodies directed against a variety of targets including human self antigens for which the normal human immune system is tolerant (Lonberg, N. et al., U.S. Pat. No. 5,569,825, U.S. Pat. No. 6,300,129 and 1994, Nature 368:856-9; Green, L. et al., 1994, Nature Genet. 7:13-21; Green, L. & Jakobovits, 1998, Exp. Med. 188:483-95; Lonberg, N. and Huszar, D., 1995, Int. Rev. Immunol. 13:65-93; Kucherlapati, et al. U.S. Pat. No. 6,713,610; Bruggemann, M. et al., 1991, Eur. J. Immunol. 21:1323-1326; Fishwild, D. et al., 1996, Nat. Biotechnol. 14:845-851; Mendez, M. et al., 1997, Nat. Genet. 15:146-156; Green, L., 1999, J. Immunol. Methods 231:11-23; Yang, X. et al., 1999,

Cancer Res. 59:1236-1243; Brüggemann, M. and Taussig, M J., Curr. Opin. Biotechnol. 8:455-458, 1997; Tomizuka et al. WO02043478). The endogenous immunoglobulin loci in such mice can be disrupted or deleted to eliminate the capacity of the animal to produce antibodies encoded by endogenous genes. In addition, companies such as Abgenix, Inc. (Freemont, Calif.) and Medarex (San Jose, Calif.) can be engaged to provide human antibodies directed against a selected antigen using technology as described above.

Preparation of immunogenic antigens, and monoclonal antibody production can be performed using any suitable technique such as recombinant protein production. The immunogenic antigens can be administered to an animal in the form of purified protein, or protein mixtures including whole cells or cell or tissue extracts, or the antigen can be formed de novo in the animal's body from nucleic acids encoding said antigen or a portion thereof. Immunization with antigen can be optionally accompanied by addition of an adjuvant, such as complete Freund's adjuvant. The immune response can be monitored over the course of the immunization protocol with plasma samples being obtained by retroorbital bleeds. The plasma can be screened by ELISA (as described below), and mice with sufficient titers of anti-MCP-1 immunoglobulin can be used for fusions. Mice can be boosted intravenously with antigen 3 days before sacrifice and removal of the spleen. It is expected that 2-3 fusions for each antigen may need to be performed. Several mice will be immunized for each antigen.

To generate hybridomas producing monoclonal CCR2 antagonist antibodies, splenocytes and lymph node cells from immunized mice can be isolated and fused to an appropriate immortalized cell line, such as a mouse myeloma cell line. The resulting hybridomas can be screened for the production of antigen-specific antibodies.

A suitable immortal cell line incapable of producing immunoglobulin chains is selected as a fusion partner, e.g., a myeloma cell line such as, but not limited to, Sp2/0 and derivative cell lines, NS1 and derivatives, especially NSO engineered NSO lines such as GS-NSO, AE-1, L.5, P3X63Ag8.653, U937, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/0 or the like, or heteromyelomas, fusion products thereof, or any cell or fusion cell derived therefrom, or any other suitable cell line as known in the art (Birch et al. 1994. Biologics 22:127-133). The fused cells (hybridomas) or recombinant cells can be isolated using selective culture conditions or other suitable known methods, and cloned by limiting dilution or cell sorting, or other known methods. Cells which produce antibodies with the desired specificity can be detected by a suitable assay (e.g., ELISA) and selected for manipulation.

Other suitable methods of generating or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260(May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590689 (Ixsys, now Applied Molecular Evolution (AME), each entirely incorporated herein by reference) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Screening antibodies for specific binding to similar proteins or fragments can also be conveniently achieved using peptide display libraries. This method involves the screening of large collections of peptides for individual members having the desired function or structure. Antibody screening using peptide display libraries is well known in the art. The displayed peptide sequences can be from 3 to 5000 or more amino acids in length, frequently from 5-100 amino acids long, and often from about 8 to 25 amino acids long. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

### Antibody Fragments

Antibody fragments can be derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. F(ab′)2, Fab, Fv and ScFv antibody fragments can all be expressed in and secreted from mammalian host cells or from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)).

In other embodiments, the antibody of is a single chain Fv fragment (scFv). See WO 93/16185; U.S. Pat. No. 5,571,894; and U.S. Pat. No. 5,587,458. Fv and sFv are species with intact combining sites, that is a VH and VL domain, that are devoid of constant regions. Typically, the VH and VL domains are cloned and re-engineered to lie within a single polypeptide and connected by a flexible linker long enough to allow interaction of the two domains within the single polypeptide. Alternatively, fusion proteins may be constructed to yield fusion of an effector protein at either the amino or the carboxy terminus of an sFv. See Antibody Engineering, 1995. ed. Borrebaeck.

### Methods of Identifying Antagonists

Antagonists of CCR2 biological activity can be identified using suitable in vitro assays and in vivo models as exemplified hereinbelow.

Binding inhibition assays can be used to identify antibodies or fragments thereof which bind CCR2 and inhibit binding of another compound such as a ligand (e.g., MCP-1, MCP-2, MCP-3 and/or MCP-4) to CCR2 or a functional variant. For example, a binding assay can be conducted in which a reduction in the binding of a ligand of CCR2 (in the presence of an antibody), as compared to binding of the ligand in the absence of the antibody, is detected or measured. A composition comprising an isolated and/or recombinant mammalian CCR2 or functional variant thereof can be contacted with the ligand and antibody simultaneously, or one after the other, in either order. A reduction in the extent of binding of the ligand in the presence of the antibody, is indicative of inhibition of binding by the antibody. For example, binding of the ligand could be decreased or abolished.

In one embodiment, direct inhibition of the binding of a ligand (e.g., a chemokine such as MCP-1/CCL2) to a mammalian CCR2 or variant thereof by an antibody or fragment is monitored. For example, the ability of an antibody to inhibit the binding of ¹²⁵I-labeled MCP-1, ¹²⁵I-labeled MCP-2, ¹²⁵I-labeled MCP-3 or ¹²⁵I-labeled MCP-4 to mammalian CCR2 can be monitored. Such an assay can be conducted using suitable cells bearing CCR2 or a functional variant thereof, such as isolated blood cells (e.g., T cells, PBMC) or a suitable cell line naturally expressing CCR2, or a cell line containing nucleic acid encoding a mammalian CCR2, or a membrane fraction from said cells, for instance.

Other methods of identifying the presence of an antibody which binds CCR2 are available, such as other suitable binding assays, or methods which monitor events which are triggered by receptor binding, including signaling function and/or stimulation of a cellular response (e.g., leukocyte trafficking).

It will be understood that the inhibitory effect of antibodies of the present invention can be assessed in a binding inhibition assay. Competition between antibodies for receptor binding can also be assessed in the method. Antibodies which are identified in this manner can be further assessed to determine whether, subsequent to binding, they act to inhibit other functions of CCR2 and/or to assess their therapeutic utility.

### Signaling Assays

The binding of a ligand or promoter, such as an agonist, to CCR2 can result in signaling by this G protein-coupled receptor, and the activity of G proteins as well as other intracellular signaling molecules is stimulated. The induction of signaling function by a compound (e.g., an antibody or fragment thereof) can be monitored using any suitable method. Such an assay can be used to identify antibody agonists of CCR2. The inhibitory activity of an antibody or functional fragment thereof or other CCR2 antagonist compound candidate can be determined using a ligand or promoter in the assay, and assessing the ability of the antibody to inhibit the activity induced by ligand or promoter.

G protein activity, such as hydrolysis of GTP to GDP, or later signaling events triggered by receptor binding, such as induction of rapid and transient increase in the concentration of intracellular (cytosolic) free calcium [Ca2+]I, can be assayed by methods known in the art or other suitable methods (see e.g., Neote, K. et al., Cell, 72: 415-425 1993); Van Riper et al., J. Exp. Med., 177: 851-856 (1993); Dahinden, C. A. et al., J. Exp. Med., 179: 751-756 (1994)).

For example, the functional assay of Sledziewski et al. using hybrid G protein coupled receptors can be used to monitor the ability a ligand or promoter to bind receptor and activate a G protein (Sledziewski et al., U.S. Pat. No. 5,284,746, the teachings of which are incorporated herein by reference).

Such assays can be performed in the presence of the antibody or fragment thereof to be assessed, and the ability of the antibody or fragment to inhibit the activity induced by the ligand or promoter is determined using known methods and/or methods described herein.

Chemotaxis and Assays of Cellular Stimulation

Chemotaxis assays can also be used to assess the ability of an antibody or functional fragment thereof to act as an antagonist of CCR2. The inhibitory activity of an antibody or functional fragment thereof or other CCR2 antagonist compound candidate to block binding of a ligand to mammalian CCR2 or functional variant thereof and inhibit chemotaxis as a function associated with binding of the ligand to the receptor is useful in that regard. These assays are based on the functional migration of cells in vitro or in vivo induced by a compound, in this case either CCL2 or another ligand capable of activating CCR2. Chemotaxis can be assessed, e.g., in an assay utilizing a 96-well chemotaxis plate, or using other art-recognized methods for assessing chemotaxis. For example, the use of an in vitro transendothelial chemotaxis assay is described by Springer et al. (Springer et al., WO 94/20142, published Sep. 15, 1994, the teachings of which are incorporated herein by reference; see also Berman et al., Immunol. Invest. 17: 625-677 (1988)). Migration across endothelium into collagen gels has also been described (Kavanaugh et al., J. Immunol., 146: 4149-4156 (1991)). Stable transfectants of mouse L1-2 pre-B cells or of other suitable host cells capable of chemotaxis can be used in chemotaxis assays, for example.

Generally, chemotaxis assays monitor the directional movement or migration of a suitable cell (such as a leukocyte (e.g., lymphocyte, eosinophil, basophil)) into or through a barrier (e.g., endothelium, a filter), toward increased levels of a compound, from a first surface of the barrier toward an opposite second surface. Membranes or filters provide convenient barriers, such that the directional movement or migration of a suitable cell into or through a filter, toward increased levels of a compound, from a first surface of the filter toward an opposite second surface of the filter, is monitored. In some assays, the membrane is coated with a substance to facilitate adhesion, such as ICAM-1, fibronectin or collagen. Such assays provide an in vitro approximation of leukocyte “homing”.

For example, one can detect or measure inhibition of the migration of cells in a suitable container (a containing means), from a first chamber into or through a microporous membrane into a second chamber which contains an antibody to be tested, and which is divided from the first chamber by the membrane. A suitable membrane, having a suitable pore size for monitoring specific migration in response to compound, including, for example, nitrocellulose, polycarbonate, is selected. For example, pore sizes of about 3-8 microns, and preferably about 5-8 microns can be used. Pore size can be uniform on a filter or within a range of suitable pore sizes.

To assess migration and inhibition of migration, the distance of migration into the filter, the number of cells crossing the filter that remain adherent to the second surface of the filter, and/or the number of cells that accumulate in the second chamber can be determined using standard techniques (e.g., microscopy). In one embodiment, the cells are labeled with a detectable label (e.g., radioisotope, fluorescent label, antigen or epitope label), and migration can be assessed in the presence and absence of the antibody or fragment by determining the presence of the label adherent to the membrane and/or present in the second chamber using an appropriate method (e.g., by detecting radioactivity, fluorescence, immunoassay). The extent of migration induced by an antibody agonist can be determined relative to a suitable control (e.g., compared to background migration determined in the absence of the antibody, compared to the extent of migration induced by a second compound (i.e., a standard), compared with migration of untransfected cells induced by the antibody).

In one embodiment, particularly for T cells, monocytes or cells expressing a mammalian CCR2, transendothelial migration can be monitored. In this embodiment, transmigration through an endothelial cell layer is assessed. To prepare the cell layer, endothelial cells can be cultured on a microporous filter or membrane, optionally coated with a substance such as collagen, fibronectin, or other extracellular matrix proteins, to facilitate the attachment of endothelial cells. Preferably, endothelial cells are cultured until a confluent monolayer is formed. A variety of mammalian endothelial cells can are available for monolayer formation, including for example, vein, artery or microvascular endothelium, such as human umbilical vein endothelial cells (Clonetics Corp, San Diego, Calif.). To assay chemotaxis in response to a particular mammalian receptor, endothelial cells of the same mammal are preferred; however endothelial cells from a heterologous mammalian species or genus can also be used.

Generally, the assay is performed by detecting the directional migration of cells into or through a membrane or filter, in a direction toward increased levels of a compound, from a first surface of the filter toward an opposite second surface of the filter, wherein the filter contains an endothelial cell layer on a first surface. Directional migration occurs from the area adjacent to the first surface, into or through the membrane, towards a compound situated on the opposite side of the filter. The concentration of compound present in the area adjacent to the second surface, is greater than that in the area adjacent to the first surface.

In one embodiment used to test for an antibody inhibitor, a composition comprising cells capable of migration and expressing a mammalian CCR2 receptor can be placed in the first chamber. A composition comprising one or more ligands or promoters capable of inducing chemotaxis of the cells in the first chamber (having chemoattractant function) is placed in the second chamber. Preferably shortly before the cells are placed in the first chamber, or simultaneously with the cells, a composition comprising the antibody to be tested is placed, preferably, in the first chamber. Antibodies or functional fragments thereof which can bind receptor and inhibit the induction of chemotaxis, by a ligand or promoter, of the cells expressing a mammalian CCR2 in this assay are inhibitors of receptor function (e.g., inhibitors of stimulatory function). A reduction in the extent of migration induced by the ligand or promoter in the presence of the antibody or fragment is indicative of inhibitory activity. Separate binding studies (see above) could be performed to determine whether inhibition is a result of binding of the antibody to receptor or occurs via a different mechanism.

In vivo assays which monitor leukocyte infiltration of a tissue, in response to injection of a compound (e.g., chemokine or antibody) in the tissue, are models of in vivo homing and measure the ability of cells to respond to a ligand or promoter by migration and chemotaxis to a site of inflammation and to assess the ability of an antibody or fragment thereof to block this migration.

In addition to the methods described, the effects of an antibody or fragment on the stimulatory function of CCR2 can be assessed by monitoring cellular responses induced by active receptor, using suitable host cells containing receptor.

### Identification of Additional Ligands and Inhibitors of Mammalian CCR2 Function

The assays described above, which can be used to assess binding and function of the antibodies and fragments of the present invention, can be adapted to identify additional ligands or other substances which bind a mammalian CCR2 or functional variant thereof, as well as inhibitors and/or promoters of mammalian CCR2 function. For example, agents having the same or a similar binding specificity as that of an antibody of the present invention or functional portion thereof can be identified by a competition assay with said antibody or portion thereof. Thus, the present invention also encompasses methods of identifying ligands of the receptor or other substances which bind a mammalian CCR2 protein, as well as inhibitors (e.g., antagonists) or promoters (e.g., agonists) of receptor function. In one embodiment, cells bearing a mammalian CCR2 protein or functional variant thereof (e.g., leukocytes, cell lines or suitable host cells which have been engineered to express a mammalian CCR2 protein or functional variant encoded by a nucleic acid introduced into said cells) are used in an assay to identify and assess the efficacy of ligands or other substances which bind receptor, including inhibitors or promoters of receptor function. Such cells are also useful in assessing the function of the expressed receptor protein or polypeptide.

According to the present invention, ligands and other substances which bind receptor, inhibitors and promoters of receptor function can be identified in a suitable assay, and further assessed for therapeutic effect Inhibitors of receptor function can be used to inhibit (reduce or prevent) receptor activity, and ligands and/or promoters can be used to induce (trigger or enhance) normal receptor function where indicated. Thus, the present invention provides a method of treating graft rejection, comprising administering an inhibitor of receptor function to an individual (e.g., a mammal).

### Pharmaceutical Compositions Comprising CCR2Antagonists

The invention includes methods for preparing pharmaceutical compositions for modulating the transcription, expression, or activity of a CCR2. Such methods comprise formulating a pharmaceutically acceptable carrier with an agent that modulates expression or activity of a CCR2. Such compositions can further include additional active agents. Thus, the invention further includes methods for preparing a pharmaceutical composition by formulating a pharmaceutically acceptable carrier with an agent that modulates expression or activity of a CCR2 and one or more additional active compounds.

Pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, histidine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), PLURONICS® and hyaluronic acid (HA).

Formulations may be designed to optimize stability of the CCR2 antagonist or, additionally, allow for sustained or extended release of the active into the bloodstream. Suitable formulations for each of type of CCR2 antagonist and route of administration may be found in, for example, “Remington: The Science and Practice of Pharmacy”, A. Gennaro, ed., 20th edition, Lippincott, Williams & Wilkins, Philadelphia, Pa., 2000.

In order for the formulations to be used for in vivo administration, they must be sterile. The formulation may be rendered sterile by filtration through sterile filtration membranes, prior to or following lyophilization and reconstitution. The therapeutic compositions herein generally are placed into a container having a sterile access port, for example, an intravenous solution bag or vial having a stopper pierceable by a hypodermic injection needle. Therapeutic compositions can be administered with medical devices known in the art.

### Methods of Treatment

The method of the invention for treatment of fibrosis in a subject includes organ specific fibrosis or systemic fibrosis. The organ specific fibrosis can be associated with at least one of lung fibrosis, liver fibrosis, kidney fibrosis, heart fibrosis, vascular fibrosis, skin fibrosis, eye fibrosis, bone marrow fibrosis or other fibrosis. The lung fibrosis can be associated with at least one of idiopathic pulmonary fibrosis, drug induced pulmonary fibrosis, asthma, sarcoidosis or chronic obstructive pulmonary disease. The liver fibrosis can be associated with at least one of cirrhosis, schistomasomiasis or cholangitis. The cirrhosis can be selected from alcoholic cirrhosis, post-hepatitis C cirrhosis, primary biliary cirrhosis. The cholangitis is sclerosing cholangitis. The kidney fibrosis can be associated with at least one of diabetic nephropathy or lupus glomeruloschelerosis. The heart fibrosis can be associated with at least one type of myocardial infarction. The vascular fibrosis can be associated with at least one of postangioplasty arterial restenosis, or atherosclerosis. The skin fibrosis can be associated with at least one of burn scarring, hypertrophic scarring, keloid, or nephrogenic fibrosing dermatopathy. The eye fibrosis can be associated with at least one of retro-orbital fibrosis, postcataract surgery or proliferative vitreoretinopathy. The bone marrow fibrosis can be associated with at least one of idiopathic myelofibrosis or drug induced myelofibrosis. The other fibrosis can be selected from Peyronie's disease, Dupuytren's contracture or dermatomyositis. The systemic fibrosis can be selected from systemic sclerosis and graft versus host disease.

### Patient Assessment

Interstitial lung disease (ILD) encompasses a diverse range of fibrotic disorders that are grouped together due to similar clinical, radiologic, physiologic, or pathologic manifestations. A more correct term, diffuse parenchymal lung diseases, is less misleading as most of these disorders affect terminal bronchioles, interstitium, and alveoli.

Although IPF is called a “pneumonia”, inflammation seems to play a relatively minor role. Environmental, genetic, or other unknown factors are thought to initially trigger alveolar epithelial cell injury, but self-perpetuating and aberrant interstitial fibroblast and mesenchymal cell proliferation (with collagen deposition and fibrosis) are thought to account for development of clinical disease. The key histologic findings are subpleuralz fibrosis with sites of fibroblast proliferation (fibroblast foci) and dense scarring, alternating with areas of normal lung tissue (heterogeneity). Scattered interstitial inflammation occurs with lymphocyte, plasma cell, and histiocyte infiltration. Cystic dilatation of peripheral alveoli (honeycombing) is found in all patients and increases with advanced disease. See Merck Manual of Diagnosis and Therapy (18th ed.), Section: Pulmonary Disorders, Subject: Interstitial Lung Diseases, Topic: Idiopathic Interstitial Pneumonias. 2006.

Diffuse lung diseases such as chronic obstructive pulmonary disease and pulmonary hypertension are excluded from the ILD classification. Patients with advanced ILD suffer from profound dyspnea and finally succumb to respiratory failure. Interstitial lung disease includes a heterogeneous group of disorders that leads to respiratory insufficiency and death in a significant number of patients. Lung transplantation is a therapeutic option in select candidates. The exact incidence and prevalence of ILD in the general population remains unknown. It is believed that ILD is far more prevalent than previously reported (5 cases per 100 000 population). A population-based study from Bernalillo County, N. Mex., reported a prevalence of 80.9 cases per 100 000 in men and 67.2 cases per 100 000 in women. Idiopathic pulmonary fibrosis (IPF) is the most common form of ILD making up about 45% of all cases in this study. Other diffuse lung diseases that may result in respiratory failure include sarcoidosis, lymphangioleiomyomatosis, Langerhan cell histiocytosis or eosinophilic granuloma, desquamative interstitial pneumonitis (DIP), nonspecific interstitial pneumonitis (NSIP), and pulmonary fibrosis associated with connective tissue disease.

Assessment of a interstitial idiopathic lung fibrosis patient (UIP/IPF, or NSIP patient) for the need of anti-CCR2 therapy can be performed at any time prior to, concurrent with, or subsequent to the symptomatic presentation of disease using methods known to those skilled in the art. Generally, methods used to diagnose pulmonary disease include severity, e.g. need for mechanical ventilation; symptoms, e.g. cough, dyspnea, fever, hemoptysis; the type of onset, gradual, acute, or subacute; underlying disease, immunodeficiency, collagen vascular disease, vasculitis; environmental exposures, asbestos, bird antigens, toxic fumes; history of medication, corticosteroids, cytotoxic agents, antibiotics; laboratory abnormalities, anemia, elevated serum eosinophil counts, antineutrophil cytoplasmic antibodies, serum precipitins against Aspergillus, rheumatoid factor; radiographic findings, normal, diffuse or localized opacities, nodular or patchy consolidation, airspace or interstitial, upper or lower lobe predominance; and pleural effusion discoverable by radiography (HRCT), ground-glass opacities, bronchiectasis, evidence of airways disease, lower lobe, subpleural, microcystic, or honeycomb change, bilateral upper lobe cysts and nodules, diffuse cystic changes bilaterally. Finally, the patient is subjected to pulmonary function tests in order to determine if the pathophysiology is obstructive, restrictive, mixed obstructive or restrictive, or normal.

Histiological findings in the IPF (UIP) patient include irregular linear opacities (reticular pattern). However, this observation may occur in collagen vascular disease, asbestosis, and chronic hypersensitivity pneumonitis. Distal lung parenchyma will have fibrosis with a honeycombing pattern in UIP. Other forms of interstitial fibrosis may occur as a result of: lymphocytic interstitial pneumonia, collagen vascular diseases, drug reactions, pneumoconiosis (asbestosis, berylliosis, silicosis, hard metal pneumoconiosis, others), sarcoidosis, Langerhans' cell histiocytosis (eosinophilic granuloma), chronic granulomatous infections, chronic aspiration, chronic hypersensitivity pneumonitis, organized chronic eosinophilic pneumonia, organized and organizing diffuse alveolar damage, chronic interstitial pulmonary edema/passive congestion, radiation (chronic), healed infectious pneumonias. The typical HRCT pattern of UIP includes bibasilar peripheral reticulation with honeycombing and traction bronchiectasis. Ground-glass opacification is not a feature of the disease, and if present, there is usually very little, some of which may actually be due to fibrosis. Two features that have been shown to be most characteristic for IPF are lower lobe honeycombing, which has an odds ratio of 5.36 for the diagnosis of IPF, and the so-called “upper lobe irregular lines,” with an odds ratio of 6.28 for IPF (Hunninghake 2003. Chest. 124:1215-1223).

Myofibroblasts are relatively absent from non-fibrotic tissue. Myofibroblasts contain smooth muscle actin fibres which gives these cells a contractile phenotype, serving to close wounds. Alpha-smooth muscle actin (aSMA) is a marker for myofibroblasts. TGFb1 is a prototypic profibrotic growth factor which has been previously shown to induce aSMA expression (Desmouliere et al. 1995 Exp Nephrol 3(2):134-9). Using cultured fibroblasts from UIP patients and fibroblasts from non-fibrotic lungs, applicants have discovered that TGFb1 induces aSMA in both non-fibrotic and fibrotic fibroblasts, however, the magnitude of induction is greater in fibrotic fibroblasts. Stimulation of the fibroblasts with PDGF induced a modest increase (less than 10-fold) in aSMA expression, but only in the fibrotic fibroblasts. Among these three, applicants data showed that only CCL2 induced an increase in aSMA expression in fibrotic fibroblasts by more than 10-fold.

Collagen deposition is a key hallmark to fibrosis and two genes, procollagen I and procollagen III, have been previously shown to be associated with UIP. Procollagen I and III proteins have been shown to be elevated in UIP samples, both in the lung and systemically (Low et al. 1992 Am Rev Respir Dis 146(3):701-6; Strieter et al. 2004 Am J Respir Crit. Care Med 170(2):133-40; Bensadoun et al. 1996. Am J Respir Crit. Care Med 154(6 Pt 1):1819-28.). Applicants have demonstrated that CCL2 induces both procollagen I and procollagen III in UIP fibroblasts. Furthermore, the magnitude of gene induction by CCL2 is comparable to that produced by both profibrotic cytokines, TGFβ1 and PDGF-AB.

The profibrotic role of TGFβ1 has been well described in a variety of tissues and organs. TGFβ1 effects are further amplified through an autocrine mechanism. Applicants observed this amplification mechanism in both non-fibrotic and fibrotic lung fibroblasts stimulated with TGFβ1 in as much as both cell types exhibited increased TGFβ1 in the presence of TGFβ1. CCL2 was also found to enhance TGFβ1 gene expression, and, as with TGFβ1, the extent of TGFβ1 gene induction by CCL2 was greater in the fibrotic fibroblasts. TGFβ receptors have been been previously found to be elevated in dermal fibroblasts from scleroderma patients (Kubo et al. 2002) Rheumatol 29(12):2558-64; Kawakami 1998. J Invest Dermatol 110(1):47-51). While UIP fibroblasts incubated with TGFβ1, PDGF and CCL2 had increases in both of the TGFβ receptor subunits analyzed over that in non-fibrotic fibroblasts, TGFβ1 mainly increased TGFβRI, and CCL2 induced both subunits indicating that the effect may not be solely due to TGFβ1 induction by CCL2.

Connective tissue growth factor (CTGF) has been shown to mediate many of the effects of TGFβ1 including collagen production. TGFβ1 induced an increase in CTGF gene expression in both fibrotic and non-fibrotic fibroblasts. Furthermore, PDGF and CCL2 induced an increase in CTGF gene expression in UIP fibroblasts.

Applicants data showed that TGFβ1, PDGF and CCL2 elevated IL13Ra1 expression in UIP fibroblasts. IL-13 is a Th2-type cytokine that is found at elevated levels in the lungs of UIP patients. IL-13 is profibrotic in vitro and in in vivo models. IL-13 induces collagen generation and proliferation of fibroblasts (Saito 2003. Int Arch Allergy Immunol 2003; 132(2):168-76; Ingram 2004 Faseb J 18(10):1132-4). A variety of murine pulmonary fibrosis models data indicate that IL13 and signaling through IL13Ra2 to be profibrotic and may also act through induction of TGFβ1 (Fichtner-Feigl 2006. Nat Med 12(1):99-106). Thus, applicants data showing that TGFβ1, PDGF-AB, and CCL2 all upregulated in IL13Ra2 expression, indicates that CCL2 may directly or indirectly render fibroblasts more sensitive to IL-13 mediated responses.

In toto, applicants have demonstrated for the first time, the hallmarks of profibrotic response by UIP fibroblasts to CCL2 and distinguished the response of UIP fibroblasts from those from non-fibrotic lungs. Previous studies have indicated that CCR2 is expressed on myofibroblast-like cells in the skin of scleroderma patients (Carulli 2005 Arthritis Rheum 52(12):3772-82) and animal models of lung fibrosis have shown an upregulation of CCR2 on fibroblasts isolated from a fibrotic, Th2-type environment and increased functionality of these cells to CCR2 ligands (Hogaboam 1999. J Immunol 1999; 163(4):2193-201.). However this is the first known study indicating a profibrotic, functional role for CCL2 on diseased lung fibroblasts.

Inhibition of CCR2-CCL2 has been shown to be beneficial in vivo models of fibrosis. Mechanistically, inhibition of fibrosis in animal models has been attributed to reduced fibrocyte recruitment and attenuated inflammation, which may or may not translate to human disease. The instant invention is based on applicants use of human fibroblasts to demonstrate that CCR2 ligand CCL2 can drive profibrotic responses and the UIP fibroblasts are hyper-responsiveness to CCL2. Thus, the data indicate that a method of blocking, inhibiting, downregulating, or antagonizing CCR2 bioactivity, especially by the antagonism of CCL2 binding to CCR2 will be effective in ameliorating the rapid and at present irreversible damage to interstitial lung tissues which result in loss of pulmonary function as applicants have established a direct profibrotic effect of CCL2 on diseased human pulmonary fibroblasts.

In the methods of the invention, a CCR2 antagonist can be administered at the time of onset of detectable markers UIP as disclosed herein, including but not limited to, CXCL5, IL-6, CCL2, IL13RA2, VEGF, IL-8 and G-CSF.

Current knowledge in the field related to diagnosis and management of IIDs, particularly IPF, indicates that the causes and initiating events leading to fibrosis are varied as is the cellular pathology (Chapman 2004 J Clin Invest 113(2): 148-157). To determine which subset of diagnosed patients might be helped by anti-CCL2 therapy, identification of biomarkers associated with CCL2 mediated disease would be of value. Studies from IPF tissue have revealed differential regulation of an up regulation of MMP-7 at the gene expression level and several proteins have been shown to be upregulated in bronchial lavage fluid and/or serum including KL-6, ENA-78, IP-10, CCL7, IL-2, IL-8, IL-10 and IL-12 (p40). However, there has to date been no correlation between these proteins and CCL2 attenuation. Several cytokines besides CCL2, such as IL-1, PDGF, Osteopontin, TNF, TGF, CCL3, CXCL8, CXCL5, CXCL12, CXCL9, CXCL10, CXCL11, have been shown to be involved in the pathogenesis of IPF. However, the relationship with CCL2 remains unclear and gene expression analysis of fibroblasts from IPF patients so far has yielded different results from different labs (See Studer et al. 2007. Proc Amer Thoracic Soc. 4(1): p. 85-9 for an overview).

Cultured fibroblasts have detectable basal levels of CCL2 and exogenously added CCL2 has recently been shown to modulate IL-6 production (Liu, X., et al. 207 Amer J Respir Cell Molec Biol. 37(1): 121-8). Therefore, CCL2 behaves as a central mediator and neutralization of CCL2 in the environment of the IPF fibroblasts will modulate expression of other genes and/or proteins.

Applicants have determined, using isolated resident fibroblasts from fibrotic patients, that lung fibroblasts from IPF patients expressed higher levels of CXCL5, IL-6, CCL2, IL13RA2, VEGF, IL-band G-CSF than non-fibrotic fibroblasts. Applicants have also determined that CNTO 888, a neutralizing CCL2 antibody, was able to block production of these proteins in cells expressing the highest levels.

An accurate diagnosis of IPF involves biopsy of lung tissue and histological analysis for unusual interstitial pneumonia. Due to the invasiveness of this procedure, it would not be practical to monitor disease progression routinely after initial diagnosis. Other non-invasive diagnoses are available but are subject to variability. Therefore a relatively non-invasive quantitative diagnostic tool is needed. Tissue fluids such as serum or bronchioalveolar lavage fluid can be sampled less invasively than surgical biopsies. Soluble proteins and cell surface markers or gene expression from collected cells could all be quantitated with existing technologies to diagnose and monitor disease progression. Monitoring tissue for these genes and/or proteins could be used to determine extent of CCL2 neutralization by CNTO 888. Therefore gene and/or protein determination for CXCL5, IL-6, CCL2, IL13RA2, VEGF, IL-8 and G-CSF from accessible peripheral tissue will provide a novel and relatively non-invasive biomarker of disease and target neutralization.

In one embodiment for assessing a patient, 5 or more ml of patient's blood can be drawn into tubes designed for nucleic acid analysis such as PAXgene Blood RNA tubes (SystemPreAnalytiX) and total RNA can be isolated and analyzed for gene expression as by any method known in the art which include the specific probes specific for at least one of CXCL5, IL-6, CCL2, IL13RA2, VEGF, IL-8 and G-CSF. Patients may be selected for therapy if any or all of the genes have expression levels greater than 2-fold of age and gender matched specimens from patients or normal subjects not displaying symptoms of patients with an idiopathic interstitial pneumonia. In another embodiment, dose selection for patients selected for therapy can be adjusted based on changes of two-fold or greater expression in any or all of the genes compared to of age and gender matched normal volunteers. In another embodiment of the method of the invention, the response to therapy can be monitored based on changes of two-fold or greater expression in any or all of the genes compared to age and gender matched normal volunteers.

In another embodiment, 5 or ml of blood can be collected from patients displaying symptoms of idiopathic interstitial pneumonia into serum collection tubes. Serum can be tested for any or all of CXCL5, IL-6, CCL2, IL13RA2, VEGF, IL-8 and G-CSF using commercially available multiplex or ELISA kits. Patients may be selected for therapy if any or all of the proteins have expression levels greater than two-fold of age and gender matched normal volunteers. In another aspect, dose selection for patients selected for therapy can be adjusted based on changes of two-fold or greater expression in any or all of the CXCL5, IL-6, CCL2, IL13RA2, VEGF, IL-8 and G-CSF compared to of age and gender matched normal volunteers. In a further aspect, the response to therapy can be monitored based on changes of two-fold or greater expression in any or all of CXCL5, IL-6, CCL2, IL13RA2, VEGF, IL-8 and G-CSF compared to age and gender matched normal volunteers.

### Routes of Administration

The route of administration is in accordance with known and accepted methods, e.g., injection or infusion by intravenous, intraperitoneal, intramuscular, intraarterial, via the portal vein; topical administration, by sustained release or extended-release means; subcutaneous injection, by transmucosal or transdermal delivery, through topical applications, nasal spray, suppository and the like, or may be administered orally.

In another aspect of the method, the administering can be by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

### Dosages

The dose of anti-CCR2 antagonist which appropriate to prevent, ameliorate, reverse, or halt the progression of chronic rejection in a patient in need thereof will be found empirically and will be dependent on the potency of the active agent, the strength of the formulation and the duration of the effective level of the agent following administration in the body of the recipient.

The course of treatment may be chronic or continuous administration in a continuous mode as opposed to an acute mode, so as to maintain the initial therapeutic effect (activity) for an extended period of time. Alternatively, the treatment may be intermittent or cyclic in nature in order to provide periods of acute antagonist activity followed by periods of lower or no antagonist activity in the body of the patient. Thus, the dosage schedule can be varied, such that the antibody is administered once, twice, three or more times per week for any number of weeks or the antibody is administered more than once (e.g., two, three, four, five, six, seven times) with administration occurring once a week, once every two, three, four, five, six, seven, eight, nine or ten weeks.

In the case of monoclonal antibody antagonist of CCR2 bioactivity, the agent will generally be administered at an amount which si based on the body weight of the recipient, e.g. between 0.1 and 100 mg/kg per course of therapy. An exemplary, non-limiting range for a therapeutically or prophylactically effective amount of an antibody administered according to the methods of the invention is 0.1-20 mg/kg, more preferably 1-10 mg/kg. In one embodiment, the anti-CCR2 or anti-CCL2 antibody can be administered by intravenous infusion at a rate of less than 10 mg/min, preferably less than or equal to 5 mg/min to reach a dose of about 1 to 500 mg/m2, preferably about 10 to 400 mg/m2, about 18 to 350 mg/m2, and more preferably, about 250-280 mg/m2. The anti-CCR2 or anti-CCL2 antibody can be administered in a single dose or in multiple doses.

### Combination Therapy

Treatment

No specific treatment has proven effective for IPF. Supportive therapy consists of O2 for hypoxemia and antibiotics for pneumonias. End-stage disease may qualify selected patients for lung transplantation. Corticosteroids and cytotoxic drugs such as cyclophosphamide (CYTOXAN), azathioprine (IMURAN) have traditionally been given to IPF patients empirically in an attempt to halt the progression of inflammation, but limited data support their efficacy. Nevertheless, it is common practice to attempt treatment with prednisone (e.g. DELTASONE) (0.5 to 1.0 mg/kg po once/day for 3 mo, tapered to 0.25 mg/kg once/day over the next 3 to 6 mo) combined with cyclophosphamide or azathioprine (1 to 2 mg/kg po once/day). Every 3 mo for 1 yr, clinical, radiographic, and physiologic responses are assessed, and drug doses are increased or decreased accordingly. Therapy is stopped if there is no objective response.

Pirfenidone, an antifibrotic agent, may stabilize pulmonary function and reduce exacerbations. Antifibrotics that inhibit collagen synthesis (relaxin), profibrotic growth factors (suramin), and endothelin-1 (an angiotensin receptor blocker) have only been demonstrated effective in vitro.

Interferon-γ-1b has shown promise when combined with prednisone in a small group of patients, but a larger double-blind multinational randomized trial found no effect on progression-free survival time, pulmonary function, or quality of life.

While having described the invention in general terms, the embodiments of the invention will be further disclosed in the following examples.

### Example 1

### Fibrotic and Non-Fibrotic Fibroblasts

In order to characterize the inherent properties of fibroblasts from UIP patients as compared to those from histopathologically non-fibrotic lung tissue, primary fibroblasts from one or the other type of tissue are assessed for markers in the unstimulated state and in response to known mediators of fibrotic pathology, e.g. TGFbeta, PDGF-AB, and CCL2.

### Fibroblast Isolation and Purification

Cell lines were provided by Dr Cory Hogaboam at the University of Michigan. All of the primary fibroblast lines were isolated as previously described (Hogaboam et al. 1999 J Immunol 163(4):2193-201). Pulmonary fibroblasts were isolated from lung biopsies taken from UIP patients (n=4) and these are referred to as “fibrotic fibroblasts”. Fibroblasts were also isolated from lung tissue taken during lung tumor resection (n=5) and these samples were confirmed to be non-fibrotic by histological analysis. These non-fibrotic tissue derived fibroblasts are referred to as “non-fibrotic fibroblasts”.

### Fibroblast Gene Expression

Human lung fibroblasts were plated into 24 well plates (Costar, Corning, N.Y.) at 100,000 cells/well and allowed to adhere for 8 hours. The cells were then washed with PBS and cultured overnight in serum free media (DMEM with 1-Glutamine, Pen/Strep). Cells were then stimulated for 24 hrs in the presence or absence of TGFb-1 (1 or 10 ng/mL), PDGF-AB (20 or 200 ng/mL) or CCL2 (1 or 10 ng/mL). TGFb1, PDGF-AB and CCL2 were purchased from R&D Systems. Supernatants were removed and RNA was subsequently isolated using RNeasy Plus Mini-Kits (QIAGEN, Valencia, Calif.) as per manufacturer's instructions and RNA was reverse transcribed into cDNA using TaqManâ Reverse Transcription Reagents (Applied Biosystems, Foster City, Calif.). Profibrotic gene expression was determined by real time PCR using the Taqmanâ Universal PCR Master Mix (Applied Biosystems) and pre-developed Taqmanâ Gene Expression Assays (Applied Biosystems) as per manufacture's instructions.

Quantitative gene expression was calculated using the comparative CT method, where CT values are determined as the threshold cycle number for which gene expression is first detected. Fold changes in gene expression for the genes of interest were first normalized to the housekeeping gene 18S, giving ACT values. Fold changes in expression between fibrotic and non-fibrotic fibroblasts were calculated as: ΔCT(non-fibrotic)−ΔCT(fibrotic)=ΔΔCT, where the non-fibrotic gene expression served as the calibrator. Fold changes in gene expression due to in vitro stimulation were calculated by ΔΔCT=ΔCT(unstimulated)−ΔCT(stimulated), where the unstimulated sample served as calibrator. The calculation 2-ΔΔCT then gives a relative value for final fold change when compared to calibrator.

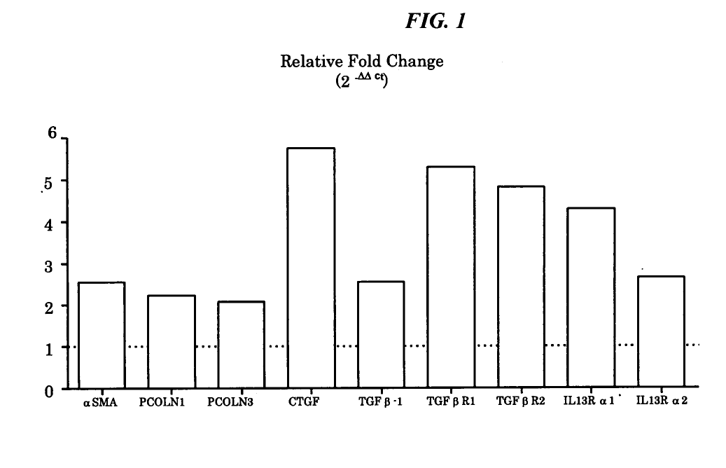

To determine if the baseline expression in profibrotic gene expression was different in UIP fibroblasts (n=3) compared to non-fibrotic (n=4) fibroblasts, gene expression was compared between the unstimulated cells from both cohorts of patients. As shown in FIG. 1., fibroblasts derived from UIP patients have greater baseline fibrotic gene expression in all of the genes analyzed.

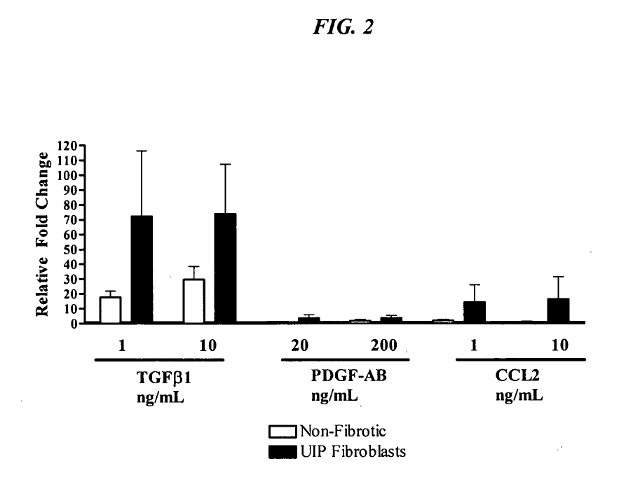

TGFb1 induced aSMA in non-fibrotic and fibrotic fibroblasts, however, the magnitude of induction was greater in fibrotic fibroblasts (FIG. 2). PDGF induced a modest increase in aSMA expression and only in the fibrotic fibroblasts. CCL2 also only induced an increase in aSMA expression in fibrotic fibroblasts.

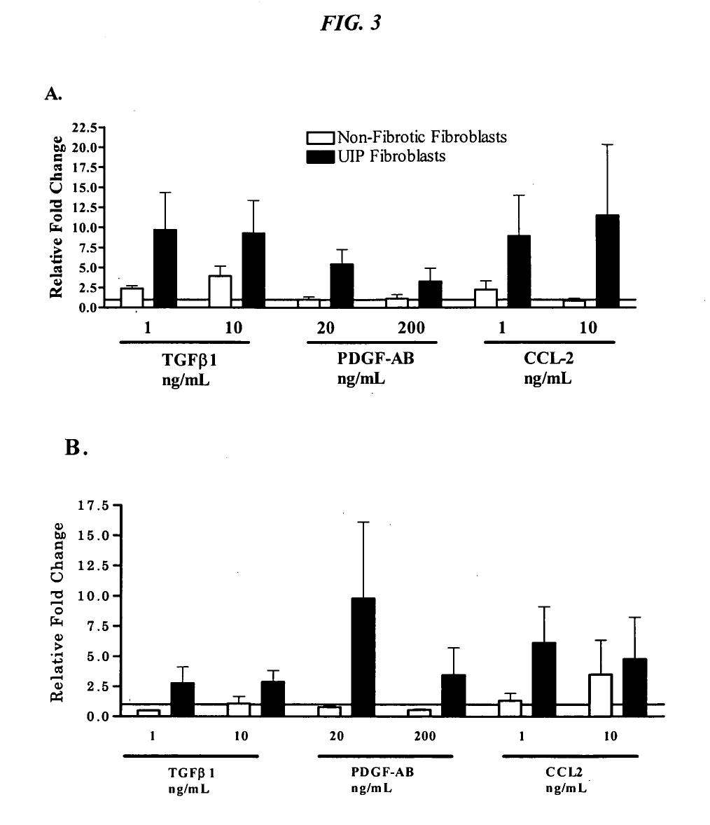

To determine whether UIP fibroblast display enhanced levels of collagen gene expression, the cells were stimulated with CCL2. FIGS. 3A and B shows that CCL2 induces both procollagen I and procollagen III in UIP fibroblasts. Furthermore, the extent of gene induction by CCL2 is comparable to that produced by either TGFb1 and PDGF-AB.

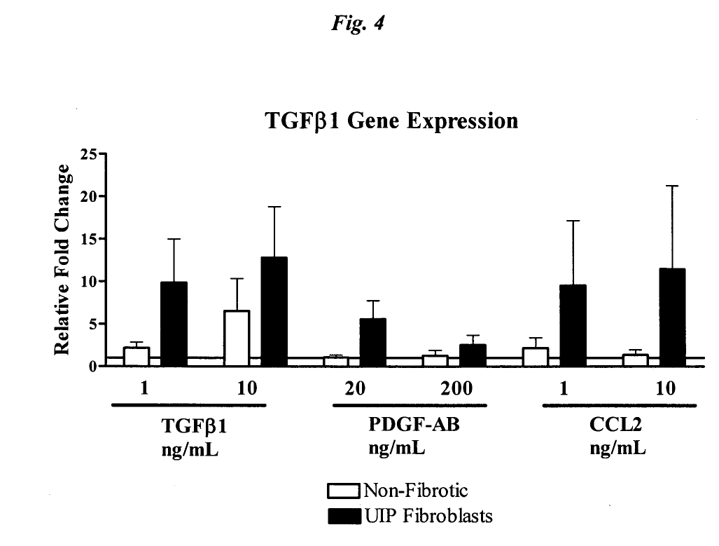

The profibrotic role of TGFb1 has been well described. TGFb1, PDGF and CCL2-induced TGFb1 gene expression (FIG. 4). TGFb1 induction by an autocrine loop is known and is supported by the present data with both the non-fibrotic and fibrotic fibroblasts. This experiment further demonstrates that CCL2 also enhances TGFb1 gene expression. As with TGFb1, the extent of TGFb1 gene induction by CCL2 was greater in the fibrotic fibroblasts.

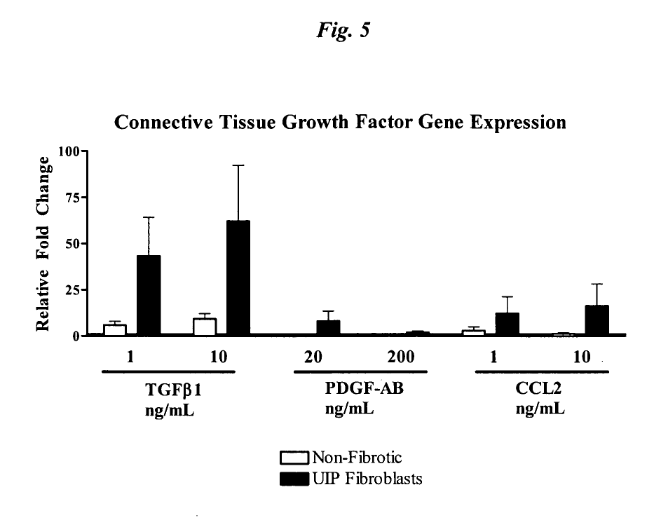

TGFb1, PDGF and CCL2-induced CTGF gene expression (FIG. 5). TGFb1 induced an increase in CTGF gene expression in both fibrotic and non-fibrotic fibroblasts. Furthermore, low-dose PDGF and CCL2 induced an increase in CTGF gene expression in UIP fibroblasts.

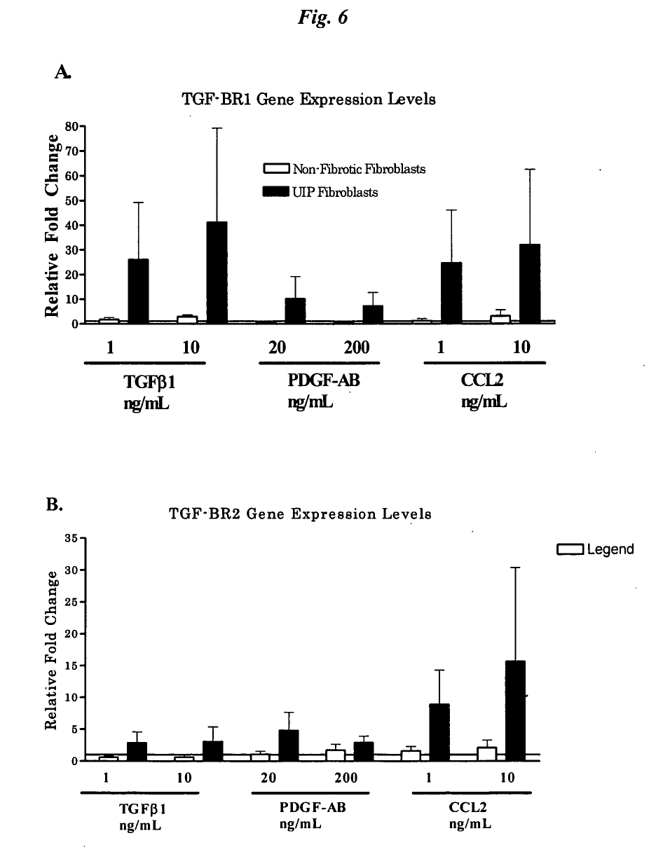

TGFb1, PDGF and CCL2-induced both TGFbRI and TGFbRII subunit gene expression is shown in FIGS. 6A and B. TGFb1 had a greater effect on TGFbRI while CCL2 induced both with a somewhat greater increase in TGFbRII.

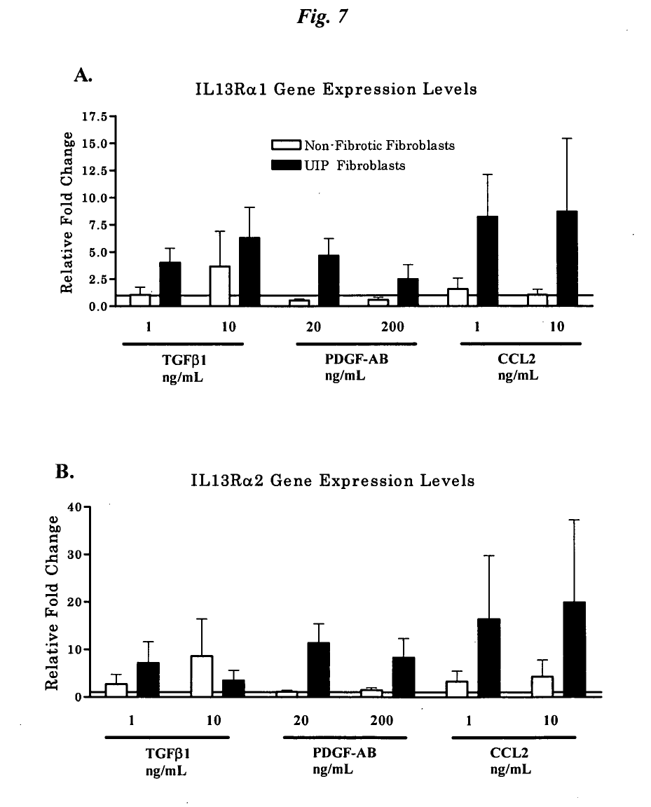

TGFb1, PDGF and CCL2-induced IL13Ra1 and IL13Ra2 gene expression is shown in FIGS. 7A and B. TGFb1, PDGF and CCL2 upregulated in IL13Ra1 expression in UIP fibroblasts. Signaling through IL13Ra2 has been recently demonstrated to be profibrotic through induction of TGFb1. All three mediators induced an upregulation in IL13Ra2 expression, thereby potentially rendering these cells more sensitive to IL-13 mediated responses.

### Example 2

Lung tissue from IPF and non-fibrotic patients was minced and put into a T75 cm tissue culture flask with 20 ml of media (DMEM w/15% FCS, 1% PSA, & L-Glutamine) Media was changed twice a week until cell colonies formed. Cells were detached and passaged. Experiments were performed after passage number five.

RNA isolation
Fibroblasts were cultured overnight in DMEM 15% FCS 1% Glutamax, 1% penicillin streptomycin at 1×105 cells in 500 ul/well of a 24 well plate. Cells were cultured for 24 hrs. in DMEM without serum. The medium was changed to DMEM supplemented with human serum albumin and cultures were incubated for 24 and/or 48 hrs. To harvest RNA, cultured cells were lysed using RNeasy mini kit (Qiagen, Inc. Valencia, Calif.) as per manufacturer's instructions. The RNA quality and quantity was determined with the 2100 BioAnalyzer (Agilent Technologies, Palo Alto, Calif.).

### RT-PCR

The Reverse transcription reaction was performed as per protocol using TaqMan® reagents (Applied Biosystems, Foster City, Calif.) at 25° C. for 10 minutes, 48° C. for 30 minutes, 95° C. for 5 minutes. Real Time PCR was performed using custom ABI low-density arrays (duplicate Assays-on-Demand primers and probes) with the ABI Prism® 7900 sequence detection system. In the presence of AmpliTaq Gold DNA polymerase (Applied Biosystems, Foster City, Calif.), the reaction was incubated for 2 min at 500 C followed by 10 min at 95° C. Then the reaction was run for 40 cycles at 15 sec, 950 C and 1 min, 600 C per cycle. The endogenous control GAPDH was used to normalize the samples using the DCT method for relative quantification.

### Immunodetection

Fibroblasts were cultured overnight as described above. Supernatants were aliquoted for testing using LINCOplex Human cytokine/chemokine 30 plex panel (Millipore), ENA-78 Quantikine kit, and IL-13RA2 ELISA kit (R&D systems)

### Results:

cDNA from 7 IPF pulmonary fibroblast lines and 5 non-fibrotic pulmonary fibroblast lines were assessed at 24 hrs by RT-PCR for relative expression of CXCL5, CCL2, IL13ra2 and IL-6. Table 1 shows the relative fold expression for each IPF line over the mean dCt (targetCt−GAPDHCt) for the non-fibrotic lines.

The IPF cell line 126 had the highest expression for CXCL5, IL13ra2 and IL-6. CCL2 expression was greater than or equal to two-fold higher than in non-fibrotic cells for 6 out of 7 cell lines. CXCL5 and IL6 expression levels were greater than or equal to two-fold higher than in non-fibrotic cells for 5 cell lines and IL13ra2 expression was greater than or equal to two-fold higher than for non-fibrotic cells for 4 cell lines.

TABLE 1

| | | | | | | | | Median |
|---|---|---|---|---|---|---|---|---|
| | Line No. | | | | | | | Fold- |
| Gene | 116 | 122 | 123 | 126 | 138 | 148 | 201 | Increase |
| CXCL5 | 1 | 2 | 6 | 2752 | 5 | 22 | 1 | 5.18 |
| CCL2 | 2 | 2 | 4 | 3 | 4 | 3 | 0 | 3 |
| IL13Ra2 | 1 | 1 | 3 | 14 | 4 | 8 | 1 | |
| IL6 | 2 | 2 | 2 | 683 | 0 | 8 | 1 | 2 |

### Protein Analysis

Supernatants from 5 UIP lines and 5 non-fibrotic lines were tested for protein expression of IL-6, IL-8, CCL2, CXCL5, G-CSF and VEGF by ELISA or multiplex. Table 2 and 3 shows the protein levels in pg/ml for all cell lines tested. Fibrotic cell line 148 had the highest expression levels for IL-6, IL-8, G-CSF, CCL2, and VEGF. Cell line 126 was highest for CXCL5 (16430 pg/ml.). IL-8 expression was greater than or equal to 2 times the median non-fibrotic expression for all 5 cell lines. CCL2 and IL-6 expression was greater than or equal to two-fold the median non-fibrotic expression for 4 out of 5 cell lines. G-CSF expression was greater than or equal to two-fold the median non-fibrotic expression for 2 cell lines. VEGF expression was greater than or equal to two-fold the median non-fibrotic expression for 3 cell lines. CXCL5 expression was greater than or equal to two-fold the median non-fibrotic expression (for values above 0) for 2 cell lines. Comparing the median values for fibrotic and non-fibrotic cell lines indicates that IL8>IL6>CCL2>VEGF in relative magnitude of difference in the median between the fibrotic and no-fibrotic cells.

TABLE 2

| | Line No. | | | | | |
|---|---|---|---|---|---|---|
| Protein | 130 | 131 | 134 | 135 | 139 | Median |
| IL6 | 10 | 4 | 18 | 63 | 25 | 18 |
| IL8 | 3 | 4 | 4 | 5 | 37 | 4 |
| G-CSF | 0 | 0 | 0 | 0 | 0 | 0 |
| CCL2 | 265 | 186 | 501 | 914 | 1506 | 501 |
| VEGF | 2 | 12 | 17 | 8 | 17 | 12 |
| CXCL5 | 0 | 0 | 84 | 0 | 5 | 0 |

TABLE 3

| | Line No. | | | | | | Fibrotic/ |
|---|---|---|---|---|---|---|---|
| Protein | 116 | 117 | 123 | 126 | 148 | Median | NonFibrotic |
| IL6 | 70 | 43 | 15 | 117 | 5086 | 70 | 3.9 |
| IL8 | 20 | 25 | 26 | 560 | 3143 | 26 | 6.0 |
| G-CSF | 0 | 0 | 0 | 71 | 1457 | 0 | — |
| CCL2 | 1675 | 2050 | 847 | 1572 | 2651 | 1675 | 3.3 |
| VEGF | 17 | 26 | 8 | 94 | 712 | 26 | 2.2 |
| CXCL5 | 3 | 3 | 77 | 16430 | 470 | 77 | — |

In order to further examine the relationship between these mediators, the fibrotic cell line 126 was treated with CCL2-neutralizing antibody CNT0888 which is comprised of the heavy and light chain variable regions given by SEQ ID NO. 27 and 28, respectively, or an irrelevant isotype matched antibody and the gene and protein expression levels compared to the same cell line with no antibody present. The results for the genetic expression are shown in the Table 4 and for protein expression in Table 5.

TABLE 4

Percent of
Untreated Cell Level

| Gene | Other IgG1 | Anti-CCL2 |
|---|---|---|
| CXCL5 | 59 | <1 |
| CCL2 | 106 | 2 |
| IL13Ra2 | 82 | 5 |
| IL6 | 98 | <1 |

TABLE 5

| | | | | Percent of Untreated | |
|---|---|---|---|---|---|
| | | | | Other | |
| Protein | Untreated | Other IgG1 | Anti-CCL2 | IgG1 | Anti-CCL2 |
| IL6 | 17442 | 19435 | 40 | 111 | <1 |
| IL8 | 13900 | 15634 | 308 | 112 | 2 |
| G-CSF | 10000 | 10000 | 1 | 100 | <1 |
| VEGF | 774 | 711 | 175 | 100 | 23 |
| CXCL5 | 12055 | 9083 | 32 | 75 | <1 |

CCL2 cannot be accurately measured in the presence of the anti-CCL2 antibody (CNT0888), although the presence of anti-CCL2 may be monitored during therapy. The highest fold reduction of protein with CNTO 888 was G-CSF (6803 fold) The lowest fold was with VEGF (4 fold): G-CSF>IL6=CXCL5>IL8>VEGF

Monitoring disease progression by lung biopsy at multiple time points would be impractical. A panel of profibrotic markers attenuated by CCL2 have been identified and can be measured ex vivo to assess CCL2 activity. Using the identified markers as surrogates would allow monitoring of antibody activity during therapy to help determine treatment options such as dose amount and timing. Secondly, we have determined a large magnitude of variability within a population of IPF pulmonary fibroblast for profibrotic gene/protein profiles which may be indicative of subsets of patients potentially responsive to anti-CCL2 therapy. Using the profibrotic panel of genes/proteins as surrogate markers of disease, provides quantitative and relatively non-invasive measurement of disease progression

1. A method for inhibiting at least one human monocyte chemoattractant protein-1 (MCP-1) in a patient having at least one form of fibrosis using at least one MCP-1 antagonist, comprising administering an MCP-1 inhibiting effective amount of at least one MCP-1 antagonists.

2. A method of claim 1, wherein said MCP-1 antagonist is selected from a small molecule and a protein.

3. A method of claim 2, wherein said small molecule is selected from indole derivatives, cyclic amine derivatives, ureido derivatives, heterocyclics, anilides, or functional pyrroles with the ability to block CCL2 binding to CCR2B, or inhibition of CCR1 or CCL2 itself.

4. A method of claim 2, wherein said protein is selected from a soluble receptor, an antibody, a peptide, a fragment thereof, or a fusion protein thereof.

5. A method of claim 4, wherein said protein further comprises a compound or protein that increases the serum half life of said protein.

6. A method of claim 4, wherein said antibody comprises at least one variable region comprising at least one heavy chain variable region and at least one light chain, said MCP-1 antibody comprising both heavy chain and light chain variable regions comprising SEQ ID NOS: 27 and 28.

7. A method of claim 4, wherein said antibody comprises at least one heavy chain variable region and at least one light chain variable region, said antibody comprising all of the heavy chain and light chain complementarity determining region (CDR) amino acid sequences of SEQ ID NOS: 6, 7, 9, 13, 14, and 16.

8. A method of claim 4, wherein said antibody comprises at least one variable region comprising at least one heavy chain and at least one light chain, said MCP-1 antibody comprising both heavy chain and light chain variable regions comprising SEQ ID NOS: 27 and 28.

9. A method of claim 4, wherein said antibody comprises at least one heavy chain variable region and at least one light chain variable region, said antibody comprising all of the heavy chain and light chain complementarity determining region (CDR) amino acid sequences of SEQ ID NOS: 6, 7, 9, 13, 14, and 16.

10. A method of claim 4, wherein said antibody comprises at least one heavy chain or light chain CDR having the amino acid sequence of at least one of SEQ ID NOS: 6, 7, 9, 13, 14, and 16.

11. A method of claim 4, wherein said antibody comprises a heavy chain or light chain variable region of at least one of SEQ ID NO: 2-5 further comprising a complementarity determining region (CDR) of a heavy or light chain or a ligand binding portion thereof selected from the group consisting of SEQ ID NO: 6-26; and, optionally functionally associated with a framework region, further optionally comprising at least CH1, hinge, CH2, or CH3 of an human immunoglobulin.

12. A method of claim 4, wherein said antibody binds MCP-1 with an affinity of at least one selected from at least 10⁻⁹M, at least 10⁻¹⁰ M, at least 10⁻¹¹ M, or at least 10⁻¹²M.

13. A method of claim 4, wherein said antibody substantially modulates at least one activity of at least one MCP-1 polypeptide.

14. A method of claim 1, wherein said small molecule or protein is provided as a composition further comprising at least one pharmaceutically acceptable carrier or diluent.

15. A method of claim 1, wherein said method further comprises administering at least one at least one compound or polypeptide selected from at least one of a detectable label or reporter, a TNF antagonist, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional product, a cytokine, or a cytokine antagonist.

16. A method according to claim 1, wherein said inhibiting effective amount is 0.001-50 mg/kilogram of said cells, tissue, organ or animal.

17. A method according to claim 1, wherein said administering is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal.

18. A method of claim 1, wherein said fibrosis is organ specific fibrosis or systemic fibrosis.

19. A method of claim 18, wherein said organ specific fibrosis is associated with at least one of lung fibrosis, liver fibrosis, kidney fibrosis, heart fibrosis, vascular fibrosis, skin fibrosis, eye fibrosis, bone marrow fibrosis or other fibrosis.

20. A method of claim 19, wherein said lung fibrosis is associated with at least one of drug induced pulmonary fibrosis, asthma, sarcoidosis or chronic obstructive pulmonary disease.

21. A method of claim 19, wherein said liver fibrosis is associated with at least one of cirrhosis, schistomasomiasis or cholangitis.

22. A method of claim 21, wherein said cirrhosis is selected from post-hepatitis C cirrhosis, primary biliary cirrhosis.

23. A method of claim 22, wherein said cholangitis is sclerosing cholangitis.

24. A method of claim 19, wherein said kidney fibrosis is associated with lupus glomeruloschelerosis.

25. A method of claim 19, where said heart fibrosis is associated with at least one type of myocardial infarction.

26. A method of claim 19, wherein said vascular fibrosis is associated with at least one of postangioplasty arterial restenosis, or atherosclerosis.

27. A method of claim 19, wherein said skin fibrosis is associated with at least one of keloid, or nephrogenic fibrosing dermatopathy.

28. A method of claim 19, wherein said eye fibrosis is associated with at least one of retro-orbital fibrosis, postcataract surgery or proliferative vitreoretinopathy.

29. A method of claim 19, wherein said bone marrow fibrosis is associated with at least one of idiopathic myelofibrosis or drug induced myelofibrosis.

30. A method of claim 19, wherein said other fibrosis is selected from Peyronie's disease, Dupuytren's contracture or dermatomyositis.

31. A method of claim 18, wherein said systemic fibrosis is selected from systemic sclerosis and graft versus host disease.

32. (canceled)


SEQUENCE LISTING (28 sequences)

SEQ ID NO: 1 - protein, 76 aa, Homo sapien
  VARIANT 1, 41, 43: Xaa = Any Amino Acid
XPDAINAPVTCCYNFTNRKISVQRLASYRRITSSKCPKEAXIXKTIVAKEICADPKQKWVQDSMDHLDKQTQTPKT

SEQ ID NO: 2 - protein, 119 aa, Homo sapien
  VARIANT 50, 52, 54, 55, 57, 58, 59: Xaa = Any Amino Acid
QVELVQSGAEVKKPGSSVKVSCKASGGTFSSYGISWVRQAPGQGLEWMGXIXPXXGXXXYAQKFQGRVTITADESTSTAYMELSSLRSEDTAVYYCARYDGIYGELDFWGQGTLVTVSS

SEQ ID NO: 3 - protein, 109 aa, Homo sapien
  VARIANT 90, 94, 95, 96, 97, 98: Xaa = Any Amino Acid
DIVLTQSPATLSLSPGERATLSCRASQSVSDAYLAWYQQKPGQAPRLLIYDASSRATGVPARFSGSGSGTDFTLTISSLEPEDFAVYYCXQYIXXXXXTFGQGTKVEIK

SEQ ID NO: 4 - protein, 120 aa, Homo sapien
QVQLVESGGGLVQPGGSLRLSCAASGFTFRSYGMSWVRQAPGKGLEWVSNIRSDGSYTYYADSVKGRFTISRDNSKNTLYLQMNSLRAEDTAVYYCARFEFTPWTYFDFWGQGTLVTVSS

SEQ ID NO: 5 - protein, 107 aa, Homo sapien
  VARIANT 89, 91, 92, 93, 96, 97: Xaa = Any Amino Acid
DIELTQPPSVSVAPGQTARISCSGDNLGKKYVYWYQQKPGQAPVLVIYDDDNRPSGIPERFSGSNSGNTATLTISGTQAEDEADYYCQXYXXXSSXXFGGGTKLTVL

SEQ ID NO: 6 - protein, 10 aa, Homo sapien
GGTFSSYGIS

SEQ ID NO: 7 - protein, 20 aa, Homo sapien
WMGGIIPIFGTANYAQKFQG

SEQ ID NO: 8 - protein, 20 aa, Homo sapien
WMGAINPLAGHTHYAQKFQG

SEQ ID NO: 9 - protein, 10 aa, Homo sapien
YDGIYGELDF

SEQ ID NO: 10 - protein, 10 aa, Homo sapien
GFTFRSYGMS

SEQ ID NO: 11 - protein, 20 aa, Homo sapien
WVSNIRSDGSYTYYADSVKG

SEQ ID NO: 12 - protein, 11 aa, Homo sapien
FEFTPWTYFDF

SEQ ID NO: 13 - protein, 12 aa, Homo sapien
RASQSVSDAYLA

SEQ ID NO: 14 - protein, 11 aa, Homo sapien
LLIYDASSRAT

SEQ ID NO: 15 - protein, 9 aa, Homo sapien
HQYIELWSF

SEQ ID NO: 16 - protein, 9 aa, Homo sapien
HQYIQLHSF

SEQ ID NO: 17 - protein, 8 aa, Homo sapien
HQYIFYPN

SEQ ID NO: 18 - protein, 11 aa, Homo sapien
SGDNLGKKYVY

SEQ ID NO: 19 - protein, 11 aa, Homo sapien
LVIYDDDNRPS

SEQ ID NO: 20 - protein, 10 aa, Homo sapien
QTYDRFSSTA

SEQ ID NO: 21 - protein, 10 aa, Homo sapien
QSYDRFSSTG

SEQ ID NO: 22 - protein, 20 aa, Homo sapien
  VARIANT 4, 6, 8, 9, 11, 12, 13: Xaa = Any Amino Acid
WMGXIXPXXGXXXYAQKFQG

SEQ ID NO: 23 - protein, 22 aa, Homo sapien
  VARIANT 10, 11, 13, 15, 19: Xaa = Any Amino Acid
WVSSIEHKWXXYXTXYAAXVKG

SEQ ID NO: 24 - protein, 8 aa, Homo sapien
  VARIANT 1, 5, 6, 7, 8: Xaa = Any Amino Acid
XQYIXXXX

SEQ ID NO: 25 - protein, 10 aa, Homo sapien
  VARIANT 2, 4, 5, 6, 9, 10: Xaa = Any Amino Acid
QXYXXXSSXX

SEQ ID NO: 26 - protein, 10 aa, Homo sapien
  VARIANT 2, 5, 9: Xaa = Any Amino Acid
GXTFXSYGXS

SEQ ID NO: 27 - protein, 119 aa, Homo sapien
QVQLVQSGAEVKKPGSSVKVSCKASGGTFSSYGISWVRQAPGQGLEWMGGIIPIFGTANYAQKFQGRVTITADESTSTAYMELSSLRSEDTAVYYCARYDGIYGELDFWGQGTLVTVSS

SEQ ID NO: 28 - protein, 109 aa, Homo sapien
  VARIANT 1: Xaa = Any Amino Acid
XIVLTQSPATLSLSPGERATLSCRASQSVSDAYLAWYQQKPGQAPRLLIYDASSRATGVPARFSGSGSGTDFTLTISSLEPEDFAVYYCHQYIQLHSFTFGQGTKVEIK